(19) United States
(12) Patent Application Publication
(10) Pub. No.: US 2018/0140250 A1
(43) Pub. Date: May 24, 2018

(54) SYSTEM AND METHOD OF PERFORMING A FIELD SOBRIETY TEST ON A USER

(71) Applicant: Jack Sweetman, Dallas, TX (US)

(72) Inventor: Jack Sweetman, Dallas, TX (US)

(21) Appl. No.: 15/359,621

(22) Filed: Nov. 23, 2016

(51) Int. Cl. A61B 5/00; A61B 5/16; G09B 7/02

(52) U.S. Cl. CPC A61B 5/4845; A61B 5/165; A61B 5/162; A61B 5/7475; G09B 7/02; A61B 5/742

(57) ABSTRACT

A system and computer implemented method for performing a field sobriety test on a user comprising a memory unit to store a base line sobriety score, and a set of program modules. The set of program modules comprises a display module, an input module and a sobriety test module. Further, the system comprises a sobriety tester module, configured to process the set of user responses. Furthermore, the sobriety tester module generates a sobriety score based on the plurality of sobriety test metrics. Furthermore, the sobriety test module classifies the user as one of sober and intoxicated based on the sobriety score being lesser than the baseline sobriety score.

### BACKGROUND OF THE INVENTION

### A. Technical Field

The present invention generally relates to the technical field of field sobriety tests.

### B. Description of Related Art

In the United States alone, approximately 34,000 fatalities and 275,000 non-fatal injuries occur every year as a result of accidents caused by drivers driving vehicles under the influence of alcohol. Recent decades have witnessed the emergence and widespread use of field sobriety tests designed to identify drivers who drive vehicles under the influence of alcohol. The field sobriety tests enables law enforcers to measure and analyze alcohol induced mental impairment in drivers. In one example, the drivers are suspects, and traffic rule violators. Further, in another example, the field sobriety tests enable the law enforcers to establish probable cause for a warrant to allow gathering of evidence. In yet another example, the field sobriety tests enables doctors to assess the condition of a patient during initial medical triage. The field sobriety testing enables the law enforcers to arrest more than 2 million drivers every year for Driving under Influence (DUI) causes. However, existing field sobriety tests have several disadvantages.

In one example, a field sobriety test comprises a walk and turn test. The walk and turn test requires a user to walk on a line drawn on the ground. The walk and turn test enables the law enforcer to understand the user's ability to have undivided attention and the user's balance. However, the result of the field sobriety test is subject to judgment of the law enforcer. The field sobriety test fails to deliver an objective result. Thus, the field sobriety test fails to objectively classify the user to one of sober or intoxicated.

In another example, a field sobriety test comprises requiring the user to breathe out into a breath analyzer. The breath analyzer analyzes breathe of the user and estimates Blood Alcohol Content (BAC) of the user. Even though the BAC result is an objective metric of the amount of alcohol in the user, the BAC result fails to indicate the alcohol induced mental impairment of the user. Further, the field sobriety test fails to detect use of a plurality of other illegal drugs. Hence, the breath analyzer fails to measure alcohol induced mental impairments of the user. Thus, the field sobriety test fails to objectively classify the user to one of sober or intoxicated. Further, the breathe analyzers are costly and unreliable.

Therefore, there is a need in the art for a system and method of a performing a field sobriety and thereby objectively classify the user into one of a sober individual and an intoxicated individual. Further, there is a need in the art for a system and method for enable law enforcers with issues such as sexual assault prevention. Further, there is a need for a system for making sobriety ratings legally valid. There is a need for a system to create statistical confidence intervals to give legitimate data about percentage chance of the user to be under a predefined BAC.

### SUMMARY OF THE INVENTION

The present invention relates to a system and method for performing a field sobriety test on a user and thereby objectively classify the user into one of a sober individual or an intoxicated individual. Further, the present invention, detects sobriety among a user by analyzing typing activities of the user. The present invention runs as a background application.

In one embodiment of the present invention, a system for performing a field sobriety test on a user comprising a memory unit to store a base line sobriety score, and a set of program modules. Further, the system detects drug use and alcohol intoxication of the user by analyzing daily activities of the user on a mobile device. The system further comprising a processor to execute the set of program modules. The set of program modules comprises a display module, an input module and a sobriety test module. The display module is configured to present the user with a set of questions. The set of questions are designed to reveal an Intelligence quotient (IQ) level of the user, and mathematical skills of the user. Further, the display module presents the user with a set of tasks. The set of tasks are designed to reveal reflex times of the user, a user reaction time and a typing speed of the user, hand eye coordination of the user, ability of the user to draw a straight line, and tendency of the user to type miss keys by more than one key on a keypad of a mobile phone. The system comprises an input module configured to receive from the user, a set of user responses to the set of questions and the set of tasks. Further, the system comprises the sobriety tester module, configured to process the set of user responses to determine a plurality of sobriety test metrics. Furthermore, the sobriety tester module generates a sobriety score based on the plurality of sobriety test metrics. Furthermore, the sobriety tester module compares the sobriety score of the user with the baseline sobriety score. Furthermore, the sobriety test module classifies the user as one of sober or intoxicated based on the sobriety score being lesser than the baseline sobriety score.

In one embodiment of the present invention, the plurality of sobriety test metrics comprises a number of typographical errors in the set of user responses, the IQ level of the user, the mathematical skills of the user, the reflex time of the user, the user reaction time, and the typing speed of the user. In another embodiment of the present invention, the set of questions comprises questions based on at least one of Stanford-Binet Intelligence scales, Woodcock-Johnson Tests of Cognitive Abilities, Raven's Progressive Matrices, and Wechsler Adult Intelligence Scale, questions based on personal details of the user, and questions based on mathematical problems. In another embodiment of the present invention, the set of tasks comprises typing a paragraph on a virtual graphical keyboard, drawing a pattern on a touchscreen, a task designed to assist in mental chronometric evaluation, and swiping in at least one of left, right, upwards and downwards directions on the touch screen. In another embodiment of the present invention, the display module presents the set of questions and the set of tasks via at least one of a Light Emitting Diode (LED screen, and a Liquid Crystal Display (LCD) screen.

In one embodiment of the present invention, a computer-implemented method of performing a field sobriety test on a user comprising storing a base line sobriety score, and a set of program modules in a memory unit. The set of program modules comprises a display module, an input module and a sobriety test module. The method comprises presenting, by a processor, the user with a set of questions. The set of questions are designed to reveal an Intelligence quotient (IQ) level of the user, and mathematical skills of the user. Further, the method comprises presenting, by a processor, the user with a set of tasks. The set of tasks are designed to reveal reflex times of the user, a user reaction time and a typing speed of the user. The method comprises receiving from the user, a set of user responses to the set of questions and the set of tasks. Further, the method comprises processing, by a processor, the set of user responses to determine a plurality of sobriety test metrics. Furthermore, the method comprises generating, by a processor, a sobriety score based on the plurality of sobriety test metrics. Furthermore, the method comprises comparing, by a processor, the sobriety score of the user with the baseline sobriety score. Furthermore, method comprises classifying, by a processor, the user as one of sober and intoxicated based on the sobriety score being lesser than the baseline sobriety score.

The application also can detect behavior on the phone. Some individuals will have strong tendencies to check Facebook and other social media applications while intoxicated, while others will reduce their phone use significantly. Some users may stop listening to music when they are drinking, others may listen to it more. To whatever degree privacy laws and phone capabilities allow, this app will track your behavior to learn you and detect when you are intoxicated. Any recurring themes of human behavior that are telling signs that something has changed.

### BRIEF DESCRIPTION OF DRAWINGS

FIG. 1 is a block diagram of an environment implemented in accordance with various embodiments of the invention.

FIG. 2 is a block diagram of a user device in accordance with various embodiments of the invention.

FIG. 3 is flow chart of a computer-implemented method of performing a field sobriety test on a user, according to yet another embodiment of the present invention.

FIG. 4 is a screenshot view of a user analysis screen according to yet another embodiment of the present invention.

FIG. 5 is a screenshot view of a user report screen according to yet another embodiment of the present invention.

FIG. 6 is flow chart of a computer-implemented method of performing a field sobriety test on a user according to yet another embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

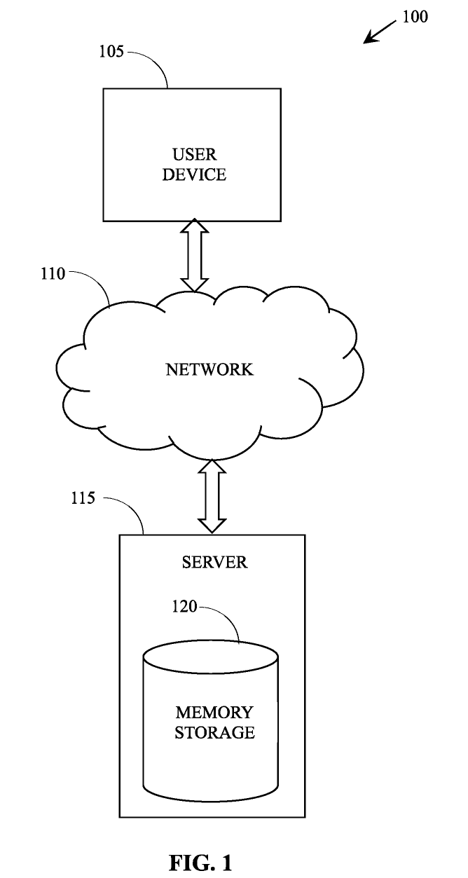

FIG. 1 is a block diagram of an environment 100 in accordance with which various embodiments of the present invention are implemented. The environment 100 comprises a user device 105, a network 110, and a server 115. The user device 105 is at least one of a tablet computer, a personal computer, a smart phone, a smart television and a laptop. In one example, the user device 105 enables the user to communicate with the server 115 via the network 110. The network 110 is one of a mobile network, a wide area network and a wireless radio network. The server 115 comprises a memory storage 120. The memory storage 120 comprises a plurality of algorithms The plurality of algorithms comprises algorithms to detect typographical errors made by the user, algorithms to calculate typing speed of the user, algorithms to calculate intelligence quotient levels of the user, algorithms to calculate a reaction time of the user, and algorithms to calculate mathematical skills of the user. In one example, the memory storage 120 stores a baseline sobriety score. The baseline sobriety score refers to a sobriety score of a field sobriety test administered to a sober individual. Further, the memory storage 120 stores a set of tasks and a set of questions. The set of questions comprises questions based on at least one of Stanford-Binet Intelligence scales, Woodcock-Johnson Tests of Cognitive Abilities, Raven's Progressive Matrices, and Wechsler Adult Intelligence Scale, questions based on personal details of the user, and questions based on mathematical problems. The set of tasks comprises typing a paragraph on a virtual graphical keyboard, drawing a pattern on a touchscreen, a task designed to assist in mental chronometric evaluation, and swiping in at least one of left, right, upwards and downwards directions on the touch screen.

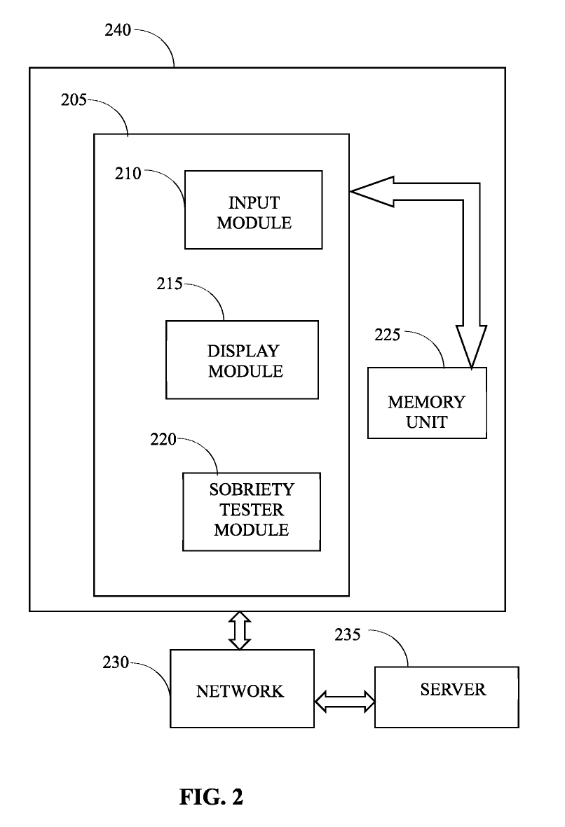

As mentioned earlier, the network 110 connects the user device 105 to the server 115. A block diagram of an exemplary implementation of the user device 105 is illustrated in FIG. 2.

Referring to FIG. 2, a user device 240 is connected to a server 235 via a network 230. The network 230 is at least one of a mobile network, a wide area network and a wireless radio network. The network 230 enables communication between the server 235 and the user device 240. The user device 240 comprises a processor 205, a memory unit 225. In one example, the memory unit 225 stores a base line sobriety score, and a set of program modules. The set of program comprises an input module 210, a display module 215 and a sobriety tester module 220. The processor 205 executes the set of program modules.

Further, the input module 210 receive from the user, a set of user inputs. The set of user inputs comprises at least one of a swipe, a tap on a touchscreen, a textual input, a gesture based input, a voice based input, and a visual input. The sobriety tester module 220 processes the set of user inputs to determine a plurality of sobriety test metrics. The plurality of sobriety test metrics comprises number of typographical errors in the set of user inputs, speed of user's speech, clarity of user's speech, steadiness of user's gesture, and ability of the user to maintain balance. The sobriety tester module 220 processes the set of user inputs with a plurality of algorithms. The plurality of algorithms are stored in the server 235. The plurality of algorithms comprises algorithms to detect degree of slur in the user's speech, algorithms to calculate speaking speed of the user, algorithms to calculate steadiness of the user's gestures, algorithms to calculate a reaction time of the user, and algorithms to calculate mathematical skills of the user. Furthermore, the sobriety tester module 220 generates a sobriety score based on the plurality of sobriety test metrics. The sobriety tester module 220 compares the sobriety score of the user with the baseline sobriety score. Further, the sobriety tester module 220 classifies the user as one of sober and intoxicated based on the sobriety score being lesser than the baseline sobriety score. The display module 215 displays classification of the user on at least one of the LCD screen, the LED screen and a computer monitor. In one example, the input module 210 continuously receives the set of user inputs and transmits the set of user inputs to the sobriety tester module 220. As a result, the sobriety tester module continuously monitors the user for intoxication.

In another example, the display module 215 present the user with a set of questions. The set of questions are designed to reveal an Intelligence quotient (IQ) level of the user, and mathematical skills of the user. Further, the display module 215 presents the user with a set of tasks. The set of tasks are designed to reveal reflex times of the user, a user reaction time and a typing speed of the user. In one example, display module 215 receives the set of questions and the set of tasks from the server 235. Further, the processor 205 executes the input module 210. Further, the input module 210 receives from the user, a set of user responses to the set of questions and the set of tasks. The sobriety tester module 220 processes the set of user responses to recalculate and update the plurality of sobriety test metrics. The plurality of sobriety test metrics further comprises number of typographical errors in the set of user responses, an IQ level of the user, a mathematical skill of the user, a reflex time of the user, a user reaction time, and a typing speed of the user. The sobriety tester module 220 processes the set of user responses with the plurality of algorithms. The plurality of algorithms further comprises algorithms to detect typographical errors made by the user, algorithms to calculate typing speed of the user, algorithms to calculate intelligence quotient levels of the user, algorithms to calculate a reaction time of the user, and algorithms to calculate mathematical skills of the user. Furthermore, the sobriety tester module 220 recalculates and updates the sobriety score based on the plurality of sobriety test metrics. The sobriety tester module 220 compares the sobriety score of the user with the baseline sobriety score. Further, the sobriety tester module 220 classifies the user as one of sober and intoxicated based on the sobriety score being lesser than the baseline sobriety score. The display module 215 displays classification of the user on at least one of the LCD screen, the LED screen and a computer monitor.

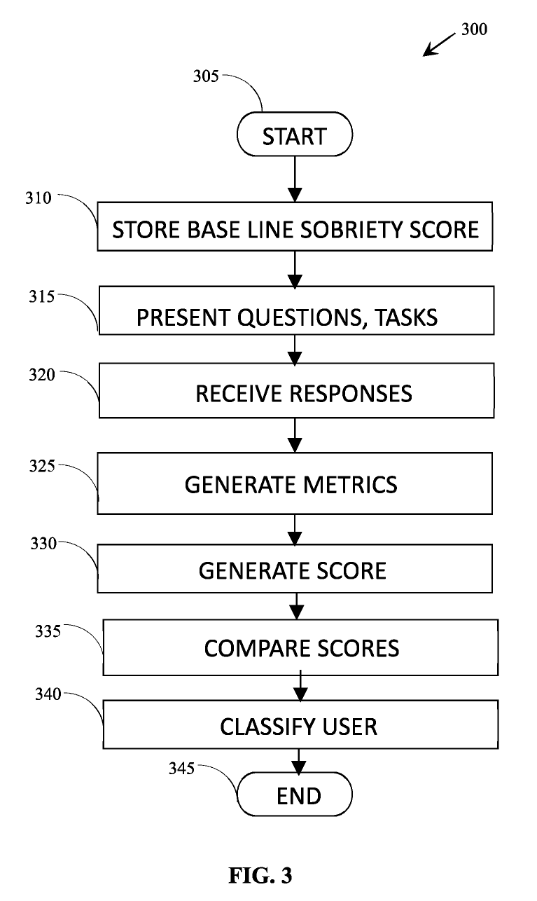

FIG. 3 is a flow chart illustrating a computer-implemented method 300 of performing a field sobriety test, according to yet another embodiment of the present invention. The method 300 is implemented in a user device. The user device is at least one of a tablet computer, a personal computer, a smart phone, a smart television and a laptop. The user device is connected to a server via a network. The network is at least one of a mobile network, a wide area network and a wireless radio network. The network enables communication between the server and the user device. The user device comprises a processor, and a memory unit. The method 300 implemented in the user device, begins at step 305.

At step 310, the memory unit stores a base line sobriety score, and a set of program modules. The set of program comprises an input module, a display module and a sobriety tester module. The processor executes the set of program modules.

At step 315, the processor via the display module, presents the user with a set of questions. The set of questions are designed to reveal an Intelligence quotient (IQ) level of the user, and mathematical skills of the user. Further, the display module presents the user with a set of tasks. The set of tasks are designed to reveal reflex times of the user, a user reaction time and a typing speed of the user. In one example, display module receives the set of questions and the set of tasks from the server. Further, the processor executes the input module.

At step 320, the processor via the input module, receives from the user, a set of user responses to the set of questions and the set of tasks. Further, the processor receives a set of user inputs.

At step 325, the processor via the sobriety tester module processes the set of user responses and the set of user inputs to determine a plurality of sobriety test metrics. The plurality of sobriety test metrics comprises number of typographical errors in the set of user responses, an IQ level of the user, a mathematical skill of the user, a reflex time of the user, a user reaction time, and a typing speed of the user. The sobriety tester module processes the set of user responses with a plurality of algorithms. The plurality of algorithms are stored in the server. The plurality of algorithms comprises algorithms to detect typographical errors made by the user, algorithms to calculate typing speed of the user, algorithms to calculate intelligence quotient levels of the user, algorithms to calculate a reaction time of the user, and algorithms to calculate mathematical skills of the user. Further, the plurality of sobriety test metrics comprises number of typographical errors in the set of user responses, the IQ level of the user, the mathematical skills of the user, the reflex time of the user, the user reaction time, and the typing speed of the user. In one example, the processor processes the set of user responses to update the plurality of sobriety test metrics.

At step 330, the processor via the sobriety tester module generates a sobriety score based on the plurality of sobriety test metrics. In one example, the processor updates the sobriety score based on the plurality of sobriety test metrics. If the app is running in the background rather than in test mode, new scores will be constantly be generated to accommodate any new actions taken on the phone.

At step 335, the processor via the sobriety tester module, compares the sobriety score of the user with the baseline sobriety score.

At step 340, the processor via the sobriety tester module classifies the user as one of sober and intoxicated based on the sobriety score being lesser than the baseline sobriety score. The display module displays classification of the user on at least one of the LCD screen, the LED screen and a computer monitor.

The method 300 ends at step 345

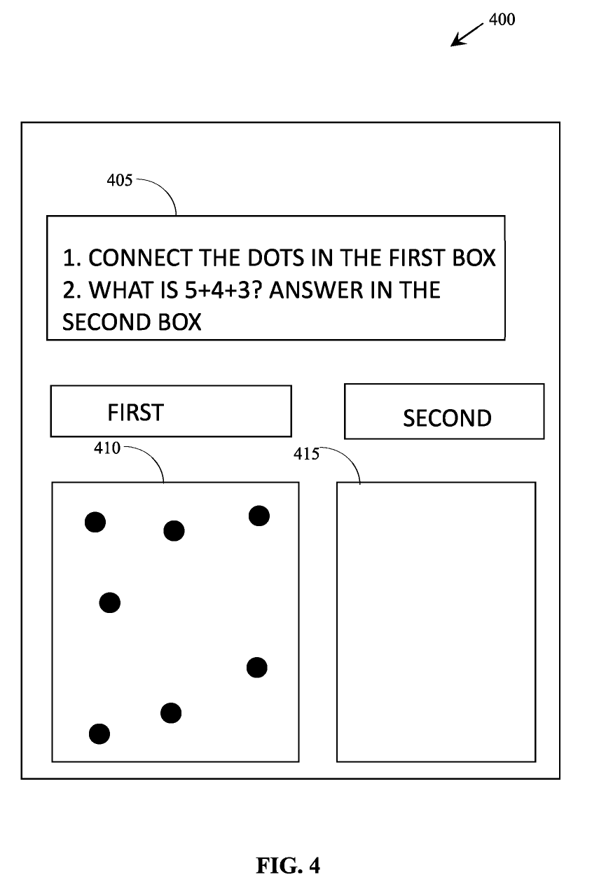

FIG. 4 is a screenshot view of a user analysis screen 400 according to yet another embodiment of the present invention. The user analysis screen 400 comprises a question box 405, a first answer box 410 and a second answer box 415. The question box 405 displays a question directed towards at least one user and a task assigned to at least one user. In one example, the question is “What is 5+4+3?” and the task is to connect a plurality of dots displayed in the first answer box 410. The user analysis screen 400 invites at least one user to connect the plurality of dots. In one example, the user analysis screen 400 is displayed on a touch screen and at least one user is enabled to connect the plurality of dots by sliding a finger over each of the plurality of dots. Further, the user analysis screen 400 invites the user to answer the question in the second answer box 415.

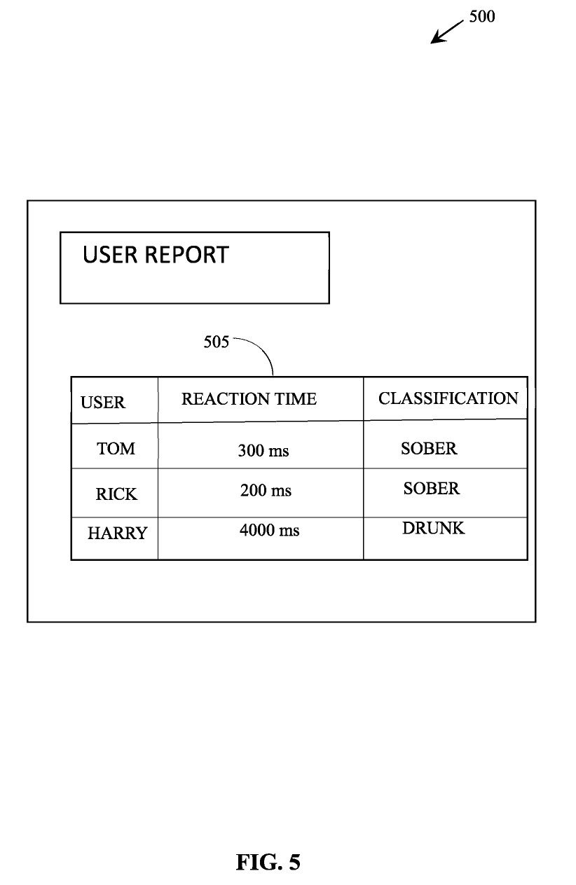

FIG. 5 is a screenshot view of a user report screen 500 according to yet another embodiment of the present invention. The user report screen 500 comprises a table 505 as displayed to a law enforcer. The table 505 comprises information about a plurality of users subjected to a field sobriety test. The information comprises the name of the users, reaction times of the user, and classification of the user.

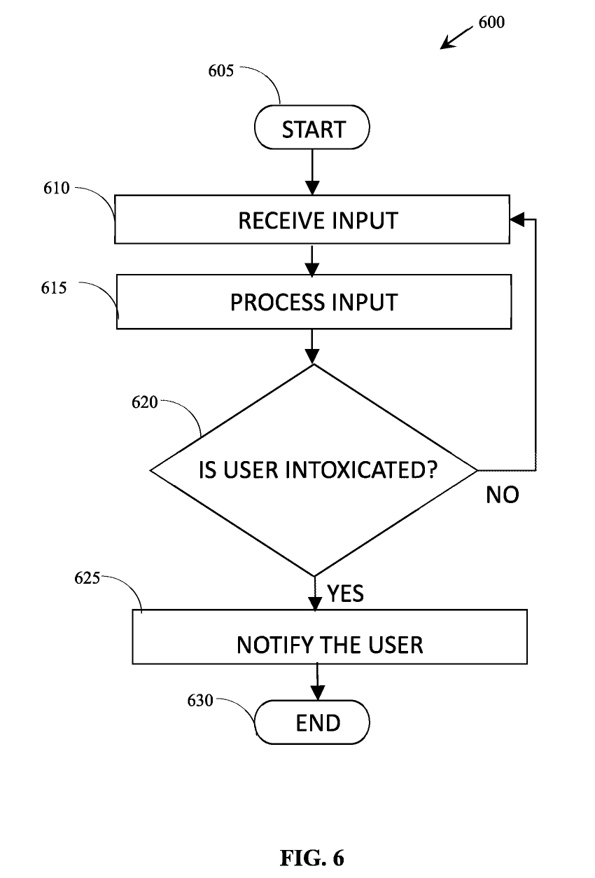

FIG. 6 is a flow chart illustrating a computer-implemented method 600 of performing a field sobriety test, according to yet another embodiment of the present invention. The method 600 is implemented in a user device. The user device is at least one of a tablet computer, a personal computer, a smart phone, a smart television and a laptop. The user device is connected to a server via a network. The network is at least one of a mobile network, a wide area network and a wireless radio network. The network enables communication between the server and the user device. The user device comprises a processor, and a memory unit. The method 600 implemented in the user device, begins at step 605.

At step 610, the processor via the input module, receives from the user, a set of user inputs. The set of user inputs comprises at least one of a swipe, a tap on a touchscreen, a textual input, a gesture based input, a voice based input, and a visual input.

At step 615, the processor via the sobriety tester module processes the set of user inputs to determine a plurality of sobriety test metrics. The plurality of sobriety test metrics comprises number of typographical errors in the set of user responses, an IQ level of the user, a mathematical skill of the user, a reflex time of the user, a user reaction time, and a typing speed of the user. The sobriety tester module processes the set of user responses with a plurality of algorithms. The plurality of algorithms are stored in the server. The plurality of algorithms comprises algorithms to detect typographical errors made by the user, algorithms to calculate typing speed of the user, algorithms to calculate intelligence quotient levels of the user, algorithms to calculate a reaction time of the user, and algorithms to calculate mathematical skills of the user. Further, the plurality of sobriety test metrics comprises number of typographical errors in the set of user responses, the IQ level of the user, the mathematical skills of the user, the reflex time of the user, the user reaction time, and the typing speed of the user. In one example, the processor processes the set of user responses to update the plurality of sobriety test metrics. Further the processor via the sobriety tester module generates a sobriety score based on the plurality of sobriety test metrics. The processor via the sobriety tester module, compares the sobriety score of the user with a baseline sobriety score. Further, the processor via the sobriety tester module classifies the user as one of sober and intoxicated based on the sobriety score being lesser than the baseline sobriety score. The display module displays classification of the user on at least one of the LCD screen, the LED screen and a computer monitor.

At step 620, if the user is intoxicated, the method 600 moves to step 625. Otherwise, the method 600 moves to step 610.

At step 625, the processor notifies the user about the user being intoxicated.

The method 600 ends at step 630

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A system for performing a field sobriety test on a user, the system comprising:
a memory unit to store a base line sobriety score, and a set of program modules;
a processor to execute the set of program modules, wherein the set of program modules comprises:
an input module, executed by the processor, configured to receive from the user, a set of user inputs, wherein the set of user inputs comprises at least one of a swipe, a tap on a touchscreen, a textual input, a gesture based input, a voice based input, and a visual input;
a sobriety tester module, executed by the processor, configured to:
process the set of user inputs to determine a plurality of sobriety test metrics,
generate a sobriety score, based on the plurality of sobriety test metrics,
compare the sobriety score of the user with the baseline sobriety score, and
classify the user as one of sober and intoxicated based on the sobriety score being lesser than the baseline sobriety score;

2. The system of claim 1, further comprising a display module, executed by the processor, configured to:
present the user with a set of questions, wherein the set of questions are designed to reveal an Intelligence quotient (IQ) level of the user, and mathematical skills of the user; and
present the user with a set of tasks, wherein the set of tasks are designed to reveal reflex times of the user, memorizing capacity of the user, organizational skills of the user, a user reaction time and a typing speed of the user.

3. The system of claim 2 wherein the input module is further configured to receive from the user a set of user responses from to the set of questions and the set of tasks.

4. The system of claim 2 wherein the sobriety tester module is further configured to:
process the set of user responses to update the plurality of sobriety test metrics;
update the sobriety score, based on the plurality of sobriety test metrics;
compare the sobriety score of the user with the baseline sobriety score; and
classify the user as one of sober and intoxicated based on the sobriety score being lesser than the baseline sobriety score.

5. The system of claim 1, wherein the plurality of sobriety test metrics comprises number of typographical errors in the set of user responses, the IQ level of the user, the mathematical skills of the user, the reflex time of the user, the user reaction time, and the typing speed of the user.

6. The system of claim 2, wherein the set of questions comprises questions based on at least one of Stanford-Binet Intelligence scales, Woodcock-Johnson Tests of Cognitive Abilities, Raven's Progressive Matrices, and Wechsler Adult Intelligence Scale, questions based on personal details of the user, and questions based on mathematical problems.

7. The system of claim 2, wherein the set of tasks comprises typing a paragraph on a virtual graphical keyboard, drawing a pattern on a touchscreen, a task designed to assist in mental chronometric evaluation, mental organization of elements, including memory and reciting the alphabet backwards and swiping in at least one of left, right, upwards and downwards directions on the touch screen.

8. The system of claim 2, wherein the display module presents the set of questions and the set of tasks via at least one of an Light Emitting Diode (LED screen, and a Liquid Crystal Display (LCD) screen.

9. A computer implemented method of performing a field sobriety test on a user, comprising:
storing a base line sobriety score in a computer system;
receiving, by the processor via the input module, a set of user inputs;
processing, by the processor, via a sobriety tester module the set of user inputs to determine a plurality of sobriety test metrics;
generating, by a processor, via the sobriety tester module a sobriety score based on the plurality of sobriety test metrics;
comparing, by the processor, via the sobriety tester module the sobriety score of the user with the baseline sobriety score; and
classifying, by the processor, via the sobriety tester module the user as one of sober and intoxicated based on the sobriety score being lesser than the baseline sobriety score.

10. The method of claim 9, further comprising:
presenting, by a processor, via a display module, a set of questions to the user, wherein the set of questions are designed to reveal an Intelligence quotient (IQ) level of the user, and mathematical skills of the user;
presenting, by the processor via the display module, a set of tasks to the user, wherein the set of tasks are designed to reveal reflex times of the user, a user reaction time and a typing speed of the user;
receiving from the user, by the processor, via the input module, a set of user responses to the set of questions and the set of tasks;
processing, by the processor, via the sobriety tester module the set of user responses to update the plurality of sobriety test metrics; and
updating, by the processor, via the sobriety tester module the sobriety score based on the plurality of sobriety test metrics.

11. The method of claim 9, wherein the plurality of sobriety test metrics comprises number of typographical errors in the set of user responses, the IQ level of the user, the mathematical skills of the user, the reflex time of the user, the user reaction time, and the typing speed of the user.

12. The method of claim 10, wherein the set of questions comprises questions based on at least one of Stanford-Binet Intelligence scales, Woodcock-Johnson Tests of Cognitive Abilities, Raven's Progressive Matrices, and Wechsler Adult Intelligence Scale, questions based on personal details of the user, and questions based on mathematical problems.

13. The method of claim 10, wherein the set of tasks comprises typing a paragraph on a virtual graphical keyboard, drawing a pattern on a touch screen, a task designed to assist in mental chronometric evaluation, and swiping in at least one of left, right, upwards and downwards directions on the touch screen.

14. The method of claim 10, wherein the display module presents the set of questions and the set of tasks via at least one of an Light Emitting Diode (LED screen, and a Liquid Crystal Display (LCD) screen.
